(19) United States
(12) Patent Application Publication
(10) Pub. No.: US 2024/0279279 A1
(43) Pub. Date: Aug. 22, 2024

(54) PROTEIN-DERIVED PEPTIDE FOR IMPROVING IMMUNITY AGAINST 2019 NOVEL CORONAVIRUS (2019-nCoV) AND USE THEREOF

(71) Applicant: ZHEJIANG A&F UNIVERSITY, Hangzhou City (CN)

(72) Inventors: Weihong MIN, Hangzhou City (CN); Fanrui ZHAO, Hangzhou City (CN)

(73) Assignee: ZHEJIANG A&F UNIVERSITY, Hangzhou City (CN)

(21) Appl. No.: 18/582,848

(22) Filed: Feb. 21, 2024

(30) Foreign Application Priority Data
Feb. 21, 2023 (CN) 202310145383.0

(51) Int. Cl. C07K 7/06; A61K 36/49; A61K 38/08; A61P 31/14

(52) U.S. Cl. CPC C07K 7/06; A61K 36/49; A61K 38/08; A61P 31/14; A61K 2236/39

(57) ABSTRACT

In some embodiments, the disclosure provides a hazelnut-derived peptide with an inhibitory activity against a 2019 novel coronavirus main protease (2019-nCoV M-pro), including an amino acid sequence of Trp-Trp-Asn-Leu-Asn (WWNLN). In other embodiments, the disclosure provides use of the hazelnut-derived peptide with an inhibitory activity against a 2019-nCoV M-pro in preparation of a drug, a health product, or a food supplementary for inhibiting a 2019-nCoV infection. In further embodiments, the derived peptide WWNLN in the disclosure has a high 2019-nCoV M-pro inhibitory activity, with an inhibition rate of 74.13%±1.93% and a half maximal inhibitory concentration (IC₅₀) value of 6.695 μM. In some embodiments, the derived peptide may be used to prepare drugs, health products, or foods supplementary that inhibit 2019-nCoV infections, and may show desirable application prospects.

### CROSS REFERENCE TO RELATED APPLICATIONS

This application claims priority to Chinese patent application number 202310145383.0, filed on Feb. 21, 2023, the disclosure of which is incorporated by reference herein in its entirety.

### FIELD OF THE DISCLOSURE

The disclosure relates generally to the fields of biology. More specifically, the disclosure relates to a hazelnut protein-derived peptide with main protease inhibitory activity of 2019 novel coronavirus (2019-nCoV M-pro) and use thereof.

### BACKGROUND

2019 novel coronavirus (2019-nCoV, also known as SARS-CoV-2) is a single-stranded and positive-sense RNA virus as well as a betacoronavirus belonging to the subgenus sarcoma virus, and is mainly transmitted through respiratory droplets and contact. Infection with the 2019-nCoV generally leads to novel coronavirus pneumonia (COVID-19), with common symptoms including respiratory symptoms, fever, cough, shortness of breath, and difficulty breathing. Some infected patients may progress to severe diseases, causing severe acute respiratory syndrome, renal failure, and even death. In view of this, it has become an urgent common issue for all mankind to curb the spread of the virus and better treat the COVID-19. It is reported that 2019-nCoV mutates rapidly, and at least 6 mutant strains have been reported. Omicron, reported in November 2021, has replaced Delta as the main popular mutant strain. Compared with Delta, Omicron mutant strains have stronger abilities of the immune evasion and transmission. The main reason is that the Omicron has 32 mutations on its spike protein, making a protective efficacy of the vaccine and neutralizing antibodies reduced or even failed.

2019 novel coronavirus main protease (2019-nCoV M-pro) is a relatively conservative protease that is responsible for catalyzing the hydrolysis of viral polyproteins (ppla and pplab) and then producing multiple functional non-structural proteins. These non-structural proteins form a replication-transcription complex to complete subsequent replication of genetic material and synthesis of structural proteins. Therefore, inhibiting the function of 2019-nCoV M-pro may interfere with replication of the virus to achieve antiviral effects. Of note, there are currently no known human-derived proteases with the same cleavage specificity as that of 2019-nCoV M-pro. This mechanism provides opportunities for drug discovery targeting the 2019-nCoV M-pro and is expected to reduce potential adverse reactions of drugs.

China is rich in hazelnut resources, and hazelnuts have a protein content of 16.2 wt % to 21.1 wt %. Moreover, hazelnut protein, as a plant-source protein, is rich in a variety of amino acids (including all 8 essential amino acids required by the human body). In addition, studies have also found that hazelnut protein hydrolyzates show a variety of biological activities, has environmental friendliness, safety, and easy absorption, and thus exhibits great development and application values. Among the biological activities, immunological activity has attracted much attention. Immunoactive peptides may enhance the phagocytosis ability of macrophages and promote the lymphocyte proliferation, thereby improving the body's ability to resist invading pathogens. Food-borne immunoactive peptides have no toxic side effects compared with traditional drugs. It is becoming a mainstream trend to improve body's immunity by consuming the food-borne immunoactive peptides. Moreover, human immunity is closely related to the 2019-nCoV infections. Based on this, the development of novel short peptides that may inhibit the 2019-nCoV M-pro may provide new ideas for the control of 2019-nCoV.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, the disclosure provides a hazelnut-derived peptide with an inhibitory activity against a 2019 novel coronavirus main protease (2019-nCoV M-pro), including an amino acid sequence of Trp-Trp-Asn-Leu-Asn (WWNLN).

In other embodiments, the disclosure provides use of the hazelnut-derived peptide with an inhibitory activity against a 2019-nCoV M-pro in preparation of a drug, a health product, or a food for inhibiting a 2019-nCoV infection.

In further embodiments, the drug, the health product, or the food further includes other ingredients and/or acceptable auxiliary materials for controlling the 2019-nCoV infection.

### BRIEF DESCRIPTION OF THE DRAWINGS

Illustrative embodiments of the disclosure are described in detail below with reference to the attached drawing figures.

FIG. 1 shows a secondary mass spectrum of the derived peptide WWNLN according to an embodiment of the disclosure.

FIG. 2 shows a 2D diagram of the molecular docking of the derived peptide WWNLN and 2019-nCoV M-pro according to an embodiment of the disclosure.

FIG. 3 shows an enzymatic reaction curve of the derived peptide WWNLN and 2019-nCoV M-pro according to an embodiment of the disclosure.

FIG. 4 shows an IC₅₀ value of the derived peptide WWNLN against 2019-nCoV M-pro according to an embodiment of the disclosure.

FIG. 5 shows an inhibition mode of the derived peptide WWNLN on 2019-nCoV M-pro according to an embodiment of the disclosure.

FIG. 6 shows a thermal stability of the derived peptide WWNLN according to an embodiment of the disclosure.

FIG. 7 shows an acid-base stability of the derived peptide WWNLN according to an embodiment of the disclosure.

FIG. 8 shows a gastrointestinal digestion stability of the derived peptide WWNLN in vitro according to an embodiment of the disclosure.

FIG. 9 shows a cytotoxicity of the derived peptide WWNLN against Vero-E6 according to an embodiment of the disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

Example 1 Screening of hazelnut-derived peptide WWNYN for 2019-nCoV M-pro inhibition

In this example, a hazelnut-derived peptide WWNYN for 2019-nCoV M-pro inhibition was extracted and screened from hazelnuts. The specific extraction and screening processes were as follows:

After hazelnuts were extruded and puffed to remove oil, hazelnut meal was obtained. The hazelnut meal and distilled water were stirred and mixed at a substrate concentration of 10 wt %, and allowed to stand in a water bath at 90° C. to 100° C. for 15 min to denature the protein. After cooling to room temperature, 1 mol/L sodium hydroxide was added dropwise to adjust the pH value to 9.0, Alcalase 2.4 L at 14,000 U/g (Novozymes) was added in hazelnut meal, and enzymatic hydrolysis was conducted with stirring at 45° C. and 10 rpm to 40 rpm for 2.5 h. During the enzymatic hydrolysis, 1 mol/L sodium hydroxide was added to maintain the pH value at 9.0. After the reaction, a hydrolyzate was quickly allowed to stand in a 90° C. water bath for 15 min to inactivate the Alcalase 2.4 L enzyme activity, then cooled to room temperature, adjusted to a neutral pH with 1 mol/L hydrochloric acid, and centrifuged at 4,500 r/min for 15 min, and a supernatant was stored at 4° C. for later use.

The supernatant, hazelnut protein hydrolysis, was separated using ultrafiltration systems with membrane pore sizes of 3,000 NMWC and 10,000 NMWC, to obtain three components with different molecular weight (<3 kDa, 3-10 kDa, >10 kDa). The hazelnut protein hydrolysis of each group were dialyzed, desalted and concentrated using a 100 Da dialysis bag: the hazelnut protein hydrolysis of each component were transferred to a 100 Da dialysis bag, and the dialysis bag was placed in a beaker containing 2 L of distilled water and stirred with magnetic force. Dialysis was conducted under the action of a dialysis machine (10 rpm to 40 rpm). When the dialysis bag was filled with liquid (about 30 h), the dialysis was completed. A resulting dialysate was concentrated using a rotary evaporator at 45° C., collected and freeze-dried at −50° C. and 10 Kpa vacuum for later use. The immune activity of four different components (<3 kDa, 3-10 kDa, >10 kDa, and hazelnut protein hydrolysis without ultrafiltration) was identified using mouse peritoneal macrophages and spleen lymphocytes. The component with the best immunological activity (<3 kDa component) was analyzed by NANO-HPLC-MS/MS. After identification, the sequence was screened by peptide chain structural characteristics and molecular simulation, and finally obtained a target sequence Trp-Trp-Asn-Tyr-Asn (WWNYN). Experiments had shown that the hazelnut-derived peptide WWNYN could inhibit 2019-nCoVM-pro (Table 3).

Example 2 Screening of derived peptide WWNLN for 2019-nCoV M-pro inhibition

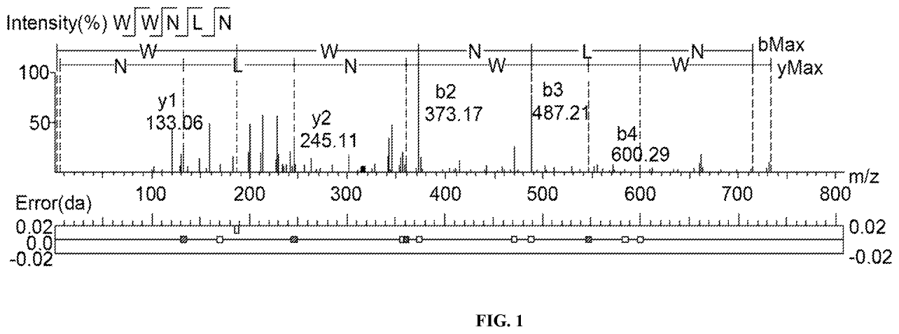

In this example, a screening process of the derived peptide WWNLN for 2019-nCoV M-pro inhibition may include: the hazelnut-derived peptide WWNYN for inhibiting 2019-nCoV M-pro (obtained by solid-phase synthesis from Jiangsu Jitai Peptide Technology Co., Ltd.) was used as a template to allow amino acid replacement and molecular docking with 2019-nCoV M-pro. The derived peptide Trp-Trp-Asn-Leu-Asn (WWNLN) with better 2019-nCoV M-pro inhibitory activity was selected based on the binding energy indicator, and a secondary mass spectrum was shown in FIG. 1.

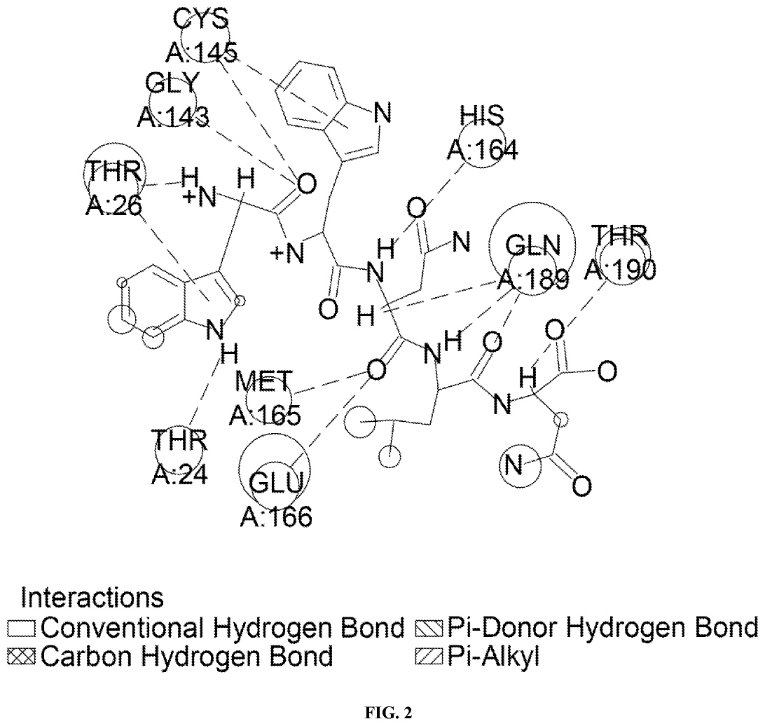

The derived peptide WWNLN for 2019-nCoV M-pro inhibition was molecularly docked with 2019-nCoV M-pro, as shown in FIG. 2 and Table 1. The results showed that after molecular docking with 2019-nCoV M-pro, WWNLN stably combined with 2019-nCoV M-pro to form 7 hydrogen bonds, and the molecular docking binding energy was −8.1 kcal/mol.

TABLE 1
Number, name, and binding energy of stable hydrogen
bonds of derived peptide WWNLN for 2019-nCoV
M-pro inhibition to 2019-nCoV M-pro.

| | | Binding energy |
|---|---|---|
| Sequence name | Binding hydrogen bond names | (kcal/mol) |
| WWNLN | Gly143, Thr26, | −8.1 |
| | Gln189, Thr24, His164, | |
| | Glu166, Cys145 | |

Example 3 Inhibitory effects of the hazelnut-derived peptide WWNYN for 2019-nCoV M-pro inhibition and the derived peptide WWNLN for 2019-nCoV M-pro inhibition on the 2019-nCoV M-pro

(1) Experiment on the Inhibitory Activity of 2019-nCoV M-Pro

2019-nCoV M-pro mother liquor, hazelnut-derived peptide WWNYN (obtained by solid-phase synthesis from Jiangsu Jitai Peptide Technology Co., Ltd.) mother liquor, and derived peptide WWNLN (obtained by solid-phase synthesis from Jiangsu Jitai Peptide Technology Co., Ltd.) mother liquor were prepared using a detection buffer (500 mM Tris, 150 mM NaCl, 1 mM EDTA, 50 v/v % glycerol); while 95 v/v % DMSO was used to prepare a stock solution of fluorescent decapeptide substrate (Mca-AVLQSGFR-K(Dnp)-K). The experimental group (containing different concentrations: 1.0, 2.5, 5.0, 10.0, 20.0 μM WWNYN/WWNLN, 20 μM fluorescent decapeptide substrate, 2 nM 2019-nCoV M-pro) and the control group (containing 20 μM fluorescent decapeptide substrate, 2 nM 2019-nCoV M-pro) solution were poured into a 96-well black microplate, and incubated in a microplate reader at 30° C. for 30 min. Microplate reader program settings were: excitation wavelength 320 nm, emission wavelength 405 nm, temperature 30° C., 150 kinetic cycles, measurement every 10 s, and record experimental data over a period of time. The enzymatic reaction curve was drawn based on the experimentally detected fluorescence intensity and enzymatic reaction time. The slope of the point on the curve represented the reaction rate at that moment, and the inhibition rate was calculated according to the formula.

$$Inhibition rate ( \% ) = ( 1 - \frac{{V}_{i}}{{V}_{0}} ) \times 100 \%$$

Vᵢ: reaction rate of adding WWNYN/WWNLN.
V₀: Reaction rate without adding WWNYN/WWNLN.
   (2) 2019-nCoV M-Pro IC₅₀ Value Calculation

The IC₅₀ value was obtained by nonlinear fitting using Graphpad Prrism8.0.1 software based on the inhibition rates at different concentrations.

(3) 2019-nCoV M-Pro Inhibition Mode Experiment

TABLE 2
12 experimental combinations of inhibition modes

| | Fluorescent decapeptide | WWNYN/WWNLN |
|---|---|---|
| | substrate concentration | concentration |
| No. | (μM) | (μM) |
| 1 | 2.5 | 0 |
| 2 | | 5 |
| 3 | | 10 |
| 4 | | 20 |
| 5 | 5.0 | 0 |
| 6 | | 5 |
| 7 | | 10 |
| 8 | | 20 |
| 9 | 10.0 | 0 |
| 10 | | 5 |
| 11 | | 10 |
| 12 | | 20 |

2019-nCoV M-pro mother liquor, hazelnut-derived peptide WWNYN (obtained by solid-phase synthesis from Jiangsu Jitai Peptide Technology Co., Ltd.) mother liquor, and derived peptide WWNLN (obtained by solid-phase synthesis from Jiangsu Jitai Peptide Technology Co., Ltd.) mother liquor were prepared using a detection buffer (500 mM Tris, 150 mM NaCl, 1 mM EDTA, 50 v/v % glycerol); while 95 v/v % DMSO was used to prepare a stock solution of fluorescent decapeptide substrate (Mca-AVLQSGFR-K(Dnp)-K). As shown in Table 2, 12 concentration combinations were designed for 4 concentrations of WWNYN/WWNLN (0, 5, 10, 20 μM) and 3 concentrations of substrate (2.5, 5.0, 10.0 μM) (the concentration of 2019-nCoV M-pro in each combination was 2 nM). The fluorescence intensity was determined under the conditions of excitation wavelength 320 nm and emission wavelength 405 nm, and inhibition mode of the inhibitor were determined based on the trend changes of Kₘ and Vₘₐₓ.

TABLE 3
Inhibitory effects of hazelnut-derived peptide WWNYN
and derived peptide WWNLN on 2019-nCoV M-pro

| Sequence | Inhibition | IC₅₀ value | |
|---|---|---|---|
| name | rate (%) | (μM) | Inhibition mode |
| WWNYN | 59.36 ± 1.05 | 19.925 | 0-10 μM: competitive inhibition |
| | | | 20 μM: non-competitive inhibition |
| WWNLN | 74.13 ± 1.93 | 6.695 | 0-10 μM: competitive inhibition |
| | | | 20 μM: non-competitive inhibition |

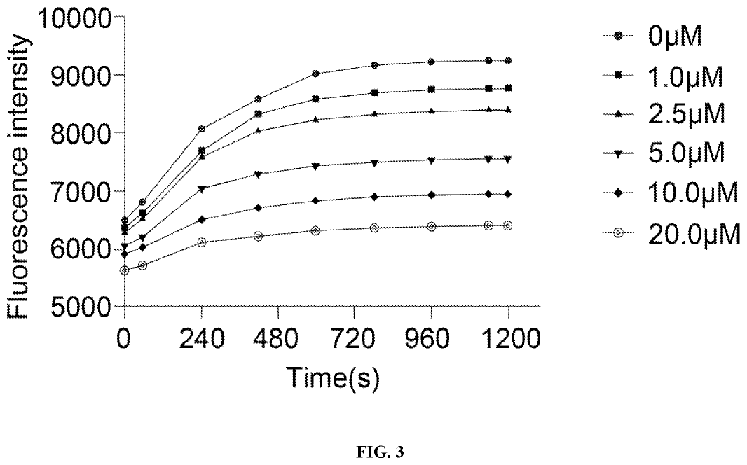

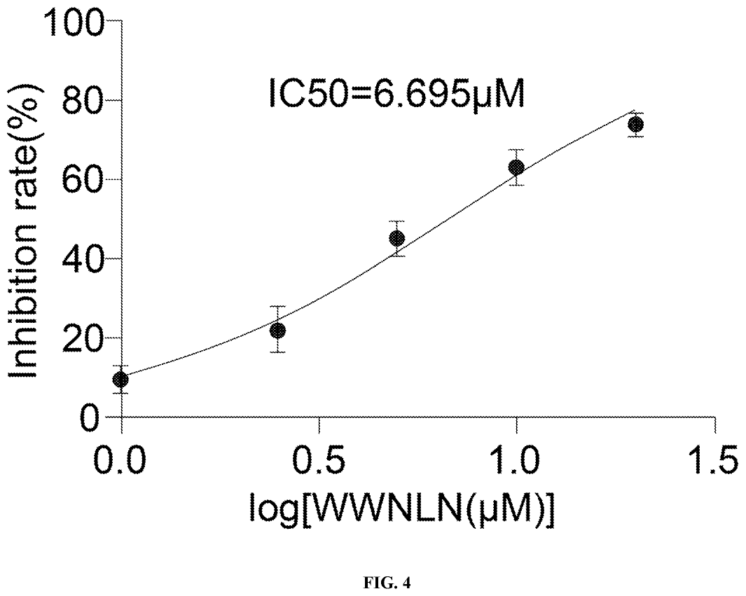

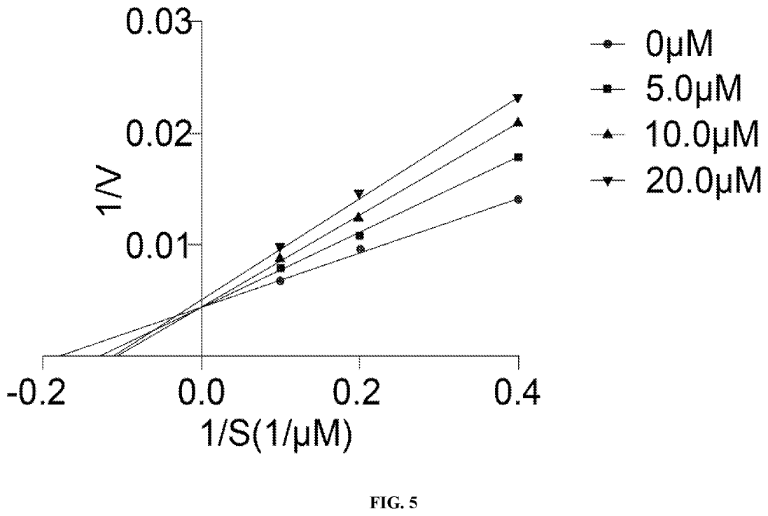

The results in FIG. 3 to FIG. 5 and Table 3 showed that the hazelnut-derived peptide WWNYN inhibited 2019-nCoV M-pro by 59.36%±1.05%, and the IC₅₀ value was 19.925 M; the derived peptide WWNLN had an inhibitory rate of 74.13%%±1.93% against 2019-nCoV M-pro, with an IC₅₀ value of 6.695 μM, indicating a better inhibitory effect. When the hazelnut-derived peptide WWNYN and the derived peptide WWNLN had an inhibitor concentration of 0 M to 10 μM, Kₘ increased but Vₘₐₓ remained unchanged, indicating competitive inhibition; when the concentration was 20 μM, Vₘₐₓ decreased, indicating non-competitive inhibition.

Example 4 Experiments on thermal stability, acid-base stability, and gastrointestinal digestion stability of the derived peptide WWNLN for 2019-nCoV M-pro inhibition

(1) Study on the Thermal Stability of the Derived Peptide WWNLN for 2019-nCoV M-Pro Inhibition

WWNLN (obtained by solid-phase synthesis from Jiangsu Jitai Peptide Technology Co., Ltd.) was diluted with distilled water into a 100 μmol/L solution, and solution that had not been treated at different temperatures was used as a blank control. The diluted WWNLN sample solution was placed in water bath conditions of 20, 40, 60, 80, and 100° C. for 2 h. The blank control and WWNLN sample solutions treated at different temperatures were injected into the RP-HPLC device for analysis through a filter membrane with a pore size of 0.22 μm. Detection wavelength was: 220 nm; column temperature was: 25° C. Mobile phase A was: lv/v % TFA+99 v/v % acetonitrile; mobile phase B was: lv/v % TFA+99 v/v % water. Elution conditions: 0 min to 25 min, 72 v/v % to 47 v/v % B (gradient elution); 25 min, 47 v/v % B (isocratic elution); 25 min to 25.1 min, 47 v/v % to 0 v/v % B (gradient elution), the column was eluted at a flow rate of 1.0 mL/min. Whether WWNLN degraded at different temperatures were determined based on the peak area and peak elution time of the liquid phase spectrum.

(2) Study on the Acid-Base Stability of the Derived Peptide WWNLN for 2019-nCoV M-Pro Inhibition

WWNLN (obtained by solid-phase synthesis from Jiangsu Jitai Peptide Technology Co., Ltd.) was diluted with distilled water into a 100 μmol/L solution, and solution that had not been treated at different pH values was used as a blank control. The pH value of the diluted WWNLN sample solution was adjusted to 2, 4, 6, 8, and 10, respectively using 1 mol/L hydrochloric acid and 1 mol/L sodium hydroxide. The blank control and WWNLN sample solutions treated with different pH levels were placed in a 37° C. water bath for 2 h. The blank control and WWNLN solutions treated at different pH values were injected into the RP-HPLC device for analysis through a filter membrane with a pore size of 0.22 m. Detection wavelength was: 220 nm; column temperature was: 25° C. Mobile phase A was: lv/v % TFA+99 v/v % acetonitrile; mobile phase B was: lv/v % TFA+99 v/v % water. Elution conditions: 0 min to 25 min, 72 v/v % to 47 v/v % B (gradient elution); 25 min, 47 v/v % B (isocratic elution); and 25 min to 25.1 min, 47 v/v % to 0 v/v % B (gradient elution), the column was eluted at a flow rate of 1.0 mL/min. Whether WWNLN degraded at different pH values was determined based on the peak area and peak elution time of the liquid phase spectrum.

(3) Analysis of the Stability of Simulated Gastrointestinal Digestion of the Derived Peptide WWNLN for 2019-nCoV M-Pro Inhibition

WWNLN (obtained by solid-phase synthesis from Jiangsu Jitai Peptide Technology Co., Ltd.) was diluted into a 100 μmol/L solution with hydrochloric acid with a pH value of 2. 0.016 g of pepsin (Beijing Solarbio Science & Technology Co., Ltd., P8390, pepsin activity 1 g: 3,000 U) was added to 1 mL of the diluted WWNLN sample solution, allowed to stand in a 37° C. water bath for 2 h, and samples were collected every 1 h and placed at 4° C. for later use. After 2 h, the WWNLN sample solution was taken out, and 1 mol/L sodium hydroxide was added to adjust the pH value of the WWNLN sample solution after pepsin enzymatic hydrolysis to 7, and 0.010 g trypsin (Beijing Solarbio Science & Technology Co., Ltd., T8150, pepsin activity 1 g: 250 U) was added, allowed to stand in a 37° C. water bath for 2 h, and samples were collected every 1 h and placed at 4° C. for later use. The WWNLN sample solution was injected into the RP-HPLC device for analysis through a filter membrane with a pore size of 0.22 μm. The elution conditions were the same as those in Example 4, part (1). Whether WWNLN was degraded after being digested by pepsin and trypsin was determined based on the peak area and peak elution time of the liquid phase spectrum.

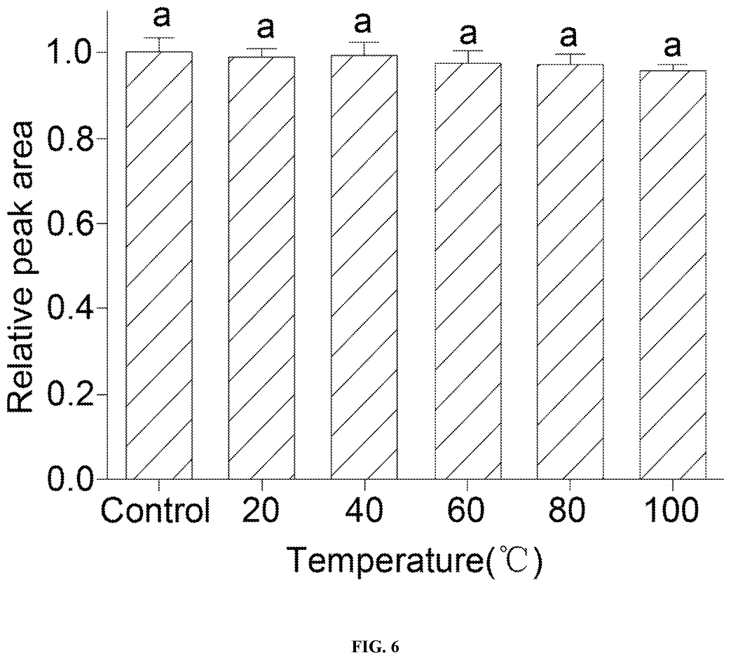

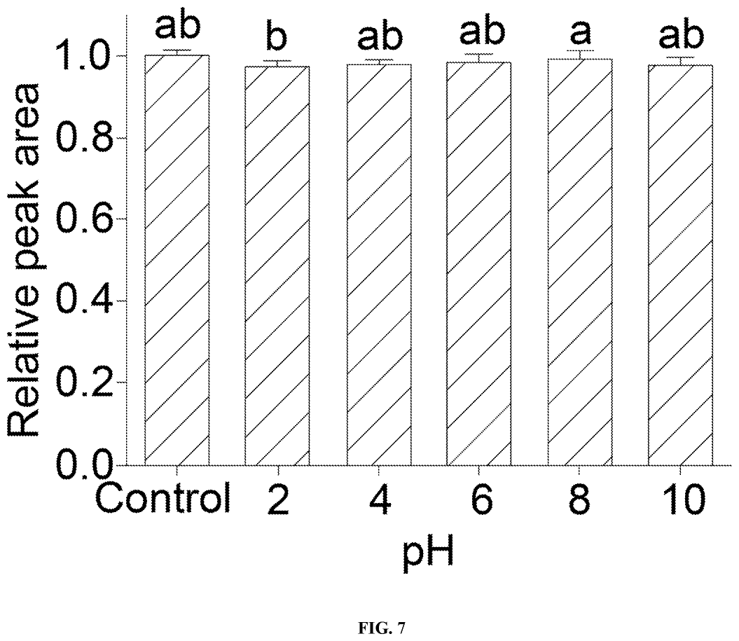

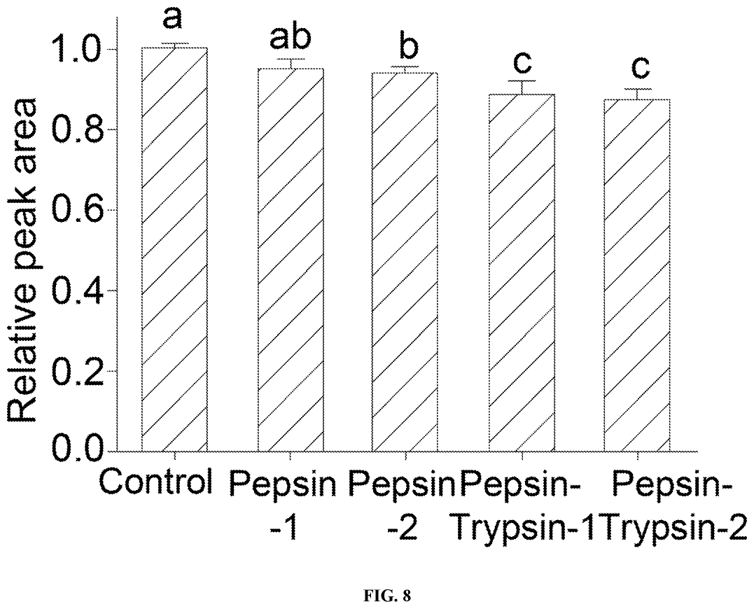

As shown in FIG. 6, compared with the blank control, the peak area and peak elution time under different temperatures of the WWNLN were not significantly different from those of the blank control, indicating that WWNLN had not been degraded and showed desirable thermal stability. As shown in FIG. 7, the peak time of WWNLN after different pH treatments was basically the same, and the peak area did not change significantly, indicating that WWNLN had not been significantly degraded in different acid-base environments. As shown in FIG. 8, after pepsin digestion for 1 h (Pepsin-1) and 2 h (Pepsin-2): compared with the blank control, the peptide was slightly degraded and the peak area was reduced. After trypsin digestion for 1 h (Pepsin-Trypsin-1) and 2 h (Pepsin-Trypsin-2): compared with the blank control, the peak area became smaller, indicating that WWNLN was degraded. Through the comparison of peak area, after digestion with pepsin and trypsin for 2 h, the retention rate of WWNLN reached 87.46%, indicating high stability.

Example 5 Cytotoxicity experiment of Vero-E6 by the derived peptide WWNLN for 2019-nCoV M-pro inhibition

The solutions (0, 25, 50, 100, 200, 400 μM; DMEM medium, Gibco Company) with different concentrations of WWNLN (obtained by solid-phase synthesis from Jiangsu Jitai Peptide Technology Co., Ltd.) and Vero-E6 cells (10⁵ cells/mL) were added in a 96-well plate, and incubated statically at 37° C. for 24 h in an incubator; the 96-well plate was taken out from the incubator, added with MTT solution to each well at a final concentration of 0.5 mg/mL, where the MTT solution was prepared using phosphate buffer (NaCl: 137 mM, KCl: 2.7 mM, Na₂HPO₄: 10 mM, KH₂PO₄: 2 mM, pH=7.2-7.4). The cells were cultured statically at 37° C. for 4 h in a CO₂ incubator (CO₂ concentration: 5 v/v %). After centrifugation at 1,000 rpm for 5 min, the supernatant was removed, 150 μL of DMSO was added to each well, and placed on a shaker at 70 rpm for 10 min. The absorbance in each well was read using a microplate reader at a wavelength of 490 nm, and the cell viability was calculated using Graphpad Prism 8.0.1. In this example, all operations were conducted in the dark after adding the MTT solution.

TABLE 4
Cytotoxicity of derived peptide WWNLN against Vero-E6

| | Derived peptide WWNLN | |
|---|---|---|
| Sequence name | concentration (μM) | Cell viability (%) |
| WWNLN | 0 | 100.00 ± 1.43 |
| | 25 | 99.59 ± 1.58 |
| | 50 | 99.29 ± 2.02 |
| | 100 | 98.86 ± 1.99 |
| | 200 | 98.36 ± 2.15 |
| | 400 | 98.03 ± 2.41 |

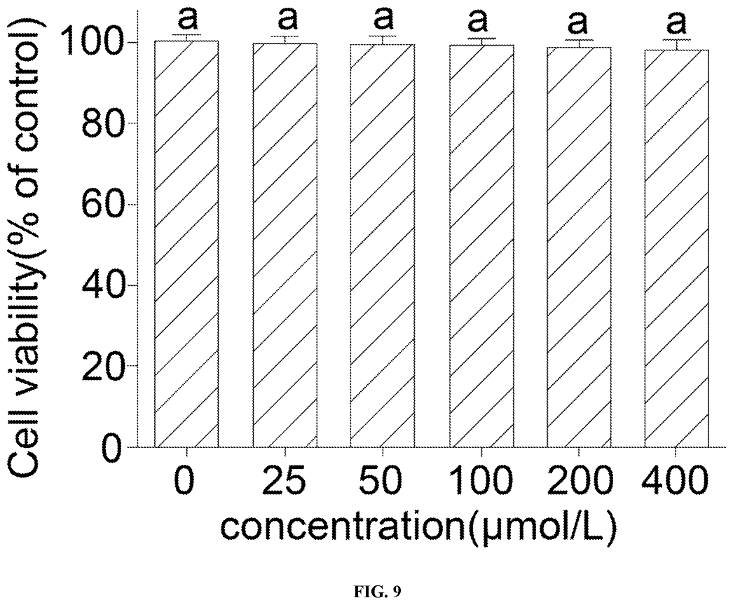

The results in FIG. 9 and Table 4 showed that in the range of 0 μM to 400 μM, the cell viability had a downward trend but showed no significant difference after analysis, indicating that the derived peptide WWNLN was not toxic to Vero-E6 cells.

Various embodiments of the disclosure may have one or more of the following effects. In some embodiments, the disclosure may provide a hazelnut-derived peptide with an inhibitory activity against a 2019-nCoV M-pro and use thereof in preparation of a drug, a health product, or a food for inhibiting a 2019-nCoV infection, which may help to solve the shortcomings of existing technologies. In other embodiments, the derived peptide may be a naturally hazelnut-derived peptide, may show high safety, thermal stability, acid and alkali resistance, and gastrointestinal digestion stability, and may have no cytotoxicity.

In further embodiments, the disclosure may propose a hazelnut-derived peptide with an inhibitory activity against a 2019 novel coronavirus main protease (2019-nCoV M-pro), including an amino acid sequence of Trp-Trp-Asn-Leu-Asn (WWNLN). In the disclosure, the hazelnut protein may be used as a raw material, enzyme-controlled hydrolysis may be conducted to prepare an enzymatic hydrolyzate, and a component with a molecular weight of less than 3 kDa may be obtained through ultrafiltration. Moreover, the component may be verified to have a positive impact on a phagocytosis ability of mouse peritoneal macrophages and a proliferation ability of spleen lymphocytes. Subsequently, a peptide structural sequence may be identified through NANO-HPLC-MS/MS. A selected hazelnut-derive peptide WWNYN may be replaced with 19 common amino acids, followed by molecular docking with the 2019-nCoV M-pro and screening in sequence to obtain a derived peptide WWNLN with a better inhibitory activity against the 2019-nCoV M-pro, showing an inhibition rate of 74.13%±1.93% and an IC₅₀ value of 6.695 M. The derived peptide WWNLN may be solid-phase chemically synthesized using a peptide synthesizer, and analyzed through thermal stability, acid-base stability, gastrointestinal tract simulation in vitro, and Vero-E6 cytotoxicity experiments. Some embodiments of the derived peptide WWNLN may show thermal stability, acid and alkali resistance, gastrointestinal digestion stability, and no cytotoxicity. The naturally derived peptide may be obtained from nut protein hydrolyzate with high safety, and may be used to prepare drugs, health products, or foods that inhibit the 2019-nCoV infection, showing desirable application prospects

Many different arrangements of the various components depicted, as well as components not shown, are possible without departing from the spirit and scope of the disclosure. Embodiments of the disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. A hazelnut-derived peptide with an inhibitory activity against a 2019 novel coronavirus main protease (2019-nCoV M-pro), comprising an amino acid sequence of Trp-Trp-Asn-Leu-Asn.

2. A method of using the hazelnut-derived peptide with an inhibitory activity against a 2019-nCoV M-pro according to claim 1 in preparation of a drug, a health product, or a food supplementary for inhibiting a 2019-nCoV infection.

3. The method according to claim 2, wherein the drug, the health product, or the food supplementary further comprises other ingredients or auxiliary materials for preventing, improving, or controlling the 2019-nCoV infection.
